(19) United States
(12) Patent Application Publication
(10) Pub. No.: US 2022/0226147 A1
(43) Pub. Date: Jul. 21, 2022

(54) ANNULAR DEVICE WITH AN INNER GRIP SURFACE FOR A MALE CONTRACEPTIVE, AND MANUFACTURING PROCESS THEREOF

(71) Applicant: Maxime LABRIT, SAINT-MÉDARD-EN-JALLES (FR)

(72) Inventor: Maxime LABRIT, SAINT-MÉDARD-EN-JALLES (FR)

(21) Appl. No.: 17/614,417

(22) PCT Filed: May 28, 2019
(86) PCT No.: PCT/FR2019/051254

§ 371 Date: Nov. 26, 2021

(51) Int. Cl. A61F 6/02

(52) U.S. Cl. CPC A61F 6/02

(57) ABSTRACT

The male contraceptive device, annular, encircles the shaft and the empty scrotum of the wearing individual of childbearing age. A rear annular edge of the device comes to abut against the base of the shaft. The inner face of the device has protrusions at least in the area of an inner adhering surface. The top of the shaft may engage against a concave upper surface portion of the device. Opposite this portion, one or more anti-slip surface portions for scrotal contact are formed in the zone separated from the shaft, in order to allow keeping a scrotal skin portion in a position in contact with the protrusions and/or with a retainer forming a slit, the rear edge raising the testicles while keeping them behind the inner volume.

### CROSS-REFERENCE TO RELATED APPLICATIONS

This application is the U.S. national phase of International Application No. PCT/FR2019/051254 filed May 28, 2019 which designated the U.S., the entire content of which is hereby incorporated by reference.

### BACKGROUND OF THE INVENTION

### Field of the Invention

The present invention relates to the domain of male contraceptive devices and comes from the category A61 F 6/02 (“Contraceptive devices for use by males”) in the meaning of the International Patent Classification. More specifically the invention relates to an annular device which does not require either taking hormones or surgery, in order to get a contraceptive effect in men. The invention also relates to a use of this annular device and to a production process thereof.

### Description of the Related Art

A device for contraception by increasing the temperature in the scrotum is known, for example, from the document U.S. Pat. No. 5,063,939 A. This device consists of a heating pocket receiving the scrotum and which attaches to the penis by a pair of attachment bands. The contraceptive effect results from the principle that heat stops spermatogenesis. The pocket has a double wall allowing insertion of a heating element electrically connected to a power source.

This type of solution is not ergonomic, and the lack of comfort makes the device unattractive. Further, the presence of an electric heating element poses some risks for the user. It should also be observed that the use of heating briefs, as shown in particular by the document CN 103919284 A, has not caused a sensation.

Another method for heating the testicles is taking hot baths in which the testicles are regularly submerged in hot water. This method is not convenient and is time-consuming because it involves a complicated process with a complex structure. Further, it does not avoid the risk of burns.

There therefore remains the need for improving the devices with which to get a male contraceptive effect in terms of ergonomics and comfort while having an effectiveness for avoiding getting temperature conditions favorable to spermatogenesis.

The condom corresponds to a widespread solution for contraception. However, it constitutes a disposable product which is relatively costly and ecologically debatable. Further, the failure rate is non-negligible, which can be attributed to the fact that it can slide unseen or can tear. It remains vulnerable to risks of damage (in combination with certain chemical contraceptive supplied to the outside or because of handling which attacks the material). Further, the condom interferes with the sexual act and can have a blocking or attenuating effect on sensations.

### SUMMARY OF THE INVENTION

To improve the situation, the invention relates to a male contraceptive device suited for encircling a shaft by having a generally annular shape around a virtual longitudinal axis, where the device comprises:
two annular edges forming two opposite axial ends of the device, a first edge called rear edge of these two edges being adapted to abut on the side (root side) of the shaft base in the mounted state of the device with the shaft encircled;
an outer face;
an inner face delimiting an inner volume and having protrusions to form an inner grip surface of the device, the inner face comprising an upper surface portion, preferably concave, intended to be in contact with the shaft on the side of its superficial dorsal vein;
a surface portion for the scrotal contact, able to be spaced/separated from the shaft and located opposite the upper surface portion, such that it serves to keep a portion of the scrotal skin between the shaft placed in the inner volume and said surface portion, while said rear edge pushes the testicles backward from the inner volume typically to a distance from this inner volume, where such rearward maintaining also corresponds to an effect of raising the testicles which cannot descend to the bottom of the scrotum (and which de facto rise upward relative to the axis of the ring).

Typically, the surface portion is formed at least in part by the inner grip surface and/or by retaining means forming a slit which extends under the inner grip surface.

The scrotal skin portion may be placed and held between the shaft placed in the inner volume and the surface portion with a retaining effect which is not uncomfortable, for thermal male contraceptive effect in the raised state of the device with the shaft and the scrotal skin of the empty scrotum encircled.

A very good compromise between comfort and retaining the scrotal skin results is obtained with tightened protrusions, in particular on an (inner) surface portion for the contact against the scrotal skin. It is therefore preferable to arrange at least 8 protrusions belonging to a single circular trajectory in a transverse section of the annular device, over a half (semi-circumference, meaning over a semi-ring) for contact with the scrotal skin. It is understood that a series of at least 15 or 16 protrusions on such a half ring (alignment of 15 or 16 protrusions on a generally semicircular path) may be provided for making the points of contact with retaining effect denser. These protrusions, which project inward, serve to multiply the discrete/discontinuous contact zones for which the spacing is reduced. Preferably at least 20 to 25 protrusions are distributed over a full circumference according to a transverse section view of the ring.

In a preferred embodiment, the two annular edges, the outer face and the inner face are formed by a single body, typically made in a single part. When retaining means forming a slit are provided, the means may also be formed from this deformable plastic body. Alternatively, such retaining means may be added onto the body, typically from the side of the lower part.

By way of these arrangements, in particular with protrusions, the device forms a penis-scrotal ring, where the inner face exerts a holding action on the scrotal skin portion in a configuration encircling both the shaft and the empty scrotum. The device provides a convenient way to move and raise the testicles, by sliding the inner face rearward against the shaft, and by inter-spacing the empty scrotum in the opposite direction between the shaft and the inner attachment surface. The rear edge of the device then acts as damn/barrier to prevent the testicles from descending, the testicles remaining near the inguinal canal. The protrusions led to an improvement in testicles holding to supra-scrotal position in order to induce an increase of the temperature of the testicles with the body as the only heat source, by limiting the sliding during motion (e.g. walking, running, jumping, etc.). They improve the general hold of the ring to the root of the shaft.

In men, induction of a temperature increase of a few degrees in the testicles held over a long time and repeated daily leads to a reduction of the production of sperm and of their mobility. The device serves to displace the testicles, from the scrotum to the root of the shaft near the outer orifice of the inguinal canal, i.e. in a warmer area. The use remains simple because placement of the penis and scrotum inside the ring without the testicles is a quick operation.

Reversibility of the method (temporary infertility/individually controlled), and the absence of side effects are further significant advantages.

Preferably, the device has a ring shape, and material making up the flexible and deformable inner face makes it possible to slide the ring around the shaft and the scrotum, without effect of striction on the shaft, so long as the ring is in contact by the rear edge against the abdominal area of the groin.

According to a particular option, the device is essentially made of elastomer, typically a silicone. This type of flexible but adhering material, presence of specific protrusions in a ring design (ring whose diameter may be preset according to a suitable size, for example by choosing between at least three sizes: 30 mm, 40 mm and 50 mm diameter or 33, 38 and 43 mm, as nonlimiting examples), allow the device to be held/maintained in place while guaranteeing a stability of the testicles at an optimal position for induction of a small temperature increase with the user body as the only heat source and by preventing the device from causing a penile restriction or any other alteration of the corpora cavernosa of the penis.

It may be advantageous, in particular for optimizing comfort, to use a rear edge perpendicular (for the most part) to the longitudinal axis.

Rows of protrusions are formed in the longitudinal direction, on the inner face, with a staggered arrangement of the protrusions in order to reduce the size of the spaces between the protrusions (the maximum inter-protrusion distance may be less than 5 or 7 mm, for example, on the inner face including the surface portion for the scrotal contact, or at least as it relates to a part forming half of the inner face). More generally, a network of protrusions is formed with rows of at least three or four protrusions along the longitudinal direction.

According to a specific feature, the inner face has an annular configuration with which to define a through passage whose radial extension measured perpendicularly to the longitudinal axis, is at least 27 mm and at most 53 mm in an undeformed state (material not stretched).

The device may have a longitudinal dimension (measured along the longitudinal axis) larger than the maximum thickness of the device.

According to a specific feature, the device has a shape which is different from the toroidal shape by a section which is oblong instead of circular (oblong section along a section made in a plane parallel to the longitudinal axis).

As a nonlimiting example, the through passage delimited by the inner face is adapted to be deformed by elastic deformation of the flexible material forming the device, where the distance between the two annular edges is preferably comprised between 15 and 24 mm at least on the side of the surface portion for the scrotal contact.

Optionally, the protrusions have the shape of bulges/nubs, the device comprising a flexible polymer material to define the nubs (a network of bulges/nubs constitutes a good compromise between the requirement of comfort and the adhesion to the skin of the penis and/or the scrotum).

According to a specific feature, the Shore A hardness of the flexible material is advantageously included between 8 and 30, for example of order 10 or 15. A low hardness significantly improves the comfort, because the material is softer, whereas the nubs or similar protrusions improve the anti-slip effect.

When the nubs have half-bead shape, they can have different diameters, for example depending on the position thereof, in order to improve the grip and give a rounder general inner shape.

It may be advantageous to form at least five bulges/nubs per square centimeter of inner grip surface. According to an option, at least eight nubs per square centimeter are provided, where this arrangement is preferably valid for the entirety of the inner face. Thus, the typically low hardness of the material combines with the irregularity of the inner grip surface in order to get a very good compromise between comfort and staying in position.

According to a specific feature, the perimeter of the nubs (and therefore the size of these nubs) increases when going away from the two annular edges of the device.

A device according to the invention may comprise one or more of the following features:
The surface portion for the scrotal contact corresponds to a surface area of at least 2 cm² and has first protrusions which project radially inward (towards the longitudinal axis).
The surface portion for the scrotal contact, which preferably correspondence to a surface area of at least 3 cm², optionally has at least one border area in which second protrusions are at least in part projecting radially outward.
The second protrusions are distributed in two rows which form all or part of the two opposite annular edges, each of the rows extending over more than a quarter of the circumference of the device.
The protrusions are distributed in parallel rows.
Each row extends over more than half of the inner circumference of the device.
A rear border zone of the protrusions and a front border zone of the protrusions are provided, where each is adjacent to or forms a part of the annular edges of the device.
The protrusions each have a height (measured perpendicularly to the longitudinal axis) of at least 0.4 mm.
The inner face has a substantially circular shape or at least as high as wide.
The surface portion for the scrotal contact has, in transverse section, substantially the same radius of curvature as that of the upper surface portion.
The surface portion for the scrotal contact has, in transverse section, a radius of curvature greater than the radius of curvature of the upper surface portion.
The surface portion for the scrotal contact has, in transverse section, a radius of curvature less than the radius of curvature of the upper surface portion.
The device presents itself in the form of a ring made as a single piece, in an elastomer, preferably a silicone-based elastomer (of course, a possible surface treatment in no way changes the fact that the device comes from a unitary implementation).
The retaining means forming a slit are part of an annular part constituting the device.
The retaining means define a housing, distinct from the inner volume, which is formed internally in a lower part of the device in order to receive the scrotal skin portion, the housing being delimited by two facing surfaces which present retaining reliefs (for example ribs or analogous protrusions defined between two grooves).
The slit is formed transversely to the longitudinal axis by having a first opening in the inner face and a second opening, opposite the first opening, in the outer face.
The slit or analogous spacing extends transversely over a portion of the periphery of the ring, by opening outward and/or inward.
The slit or spacing extends transversely to the longitudinal axis, intermediately (preferably at equal distance) between the two annular edges.
The inner opening/mouth of the spacing or slit has a general shape of a circular arc corresponding to an angular sector included between 90° and 270° around the longitudinal axis of the ring.
The radial spacing or slit may be defined between two opposite ribbed surfaces, which belong to two parallel segments of the ring, typically arranged at the same distance from the longitudinal axis.
The two parallel segments belong to a single part forming the ring.
The inner face (which extends annularly around the longitudinal axis) defines an inner diameter measured perpendicularly to the longitudinal axis, where the slit formed by the retaining means extends between two ends separated from each other by a distance greater than the inner diameter.
The device includes a structure with at least two layers in a lower part of the device, where the protrusions of the inner face are at least in part formed by a layer described as inner defining a coating, and where the slit is formed between two layers extending under the inner layer.
The slit of the ring forms a radial expansion space between two layers, where the radial expansion space extends along a set circumference portion smaller than the (whole) inner circumference of the ring.
The slit extends from one to the other between the two annular edges of the ring.
The set circumference does not extend past two thirds or three quarters of the inner circumference of the ring, and is preferably greater than one third of this inner circumference (more generally, the slit is formed over more than one quarter of a turn and less than three quarters of a turn around the longitudinal axis of the ring).
The slit, forming the radial expansion space, may be defined between two facing ribbed surfaces, one of which belongs to a first ring sub-element forming a proximal layer of the longitudinal axis and the other belonging to a second ring sub-element forming a distal layer of the longitudinal axis (where the first and second ring sub-elements are concentric).
The two sub-elements belong to a single part forming the ring.
A structure with at least two layers is provided for forming the device, where the protrusions of the inner face are at least in part formed by a coating layer.
Two layers of the structure are defined by: a base layer, having a thickness greater than a first threshold without exceeding a second threshold equal to 10 mm; a coating layer forming the inner face and for which the maximum thickness is less than or equal to the first threshold.
The coating is added onto/overmolded on the base layer.
The inner protrusions are obtained from the molding of the ring forming part; a later surface treatment may be done by a matte treatment in order to improve the adhesion/grip (without changing the profile of the protrusions).
The nubs form half-beads, optionally distributed in at least two groups of different size, which preferably cover the entire inner face of the ring; this improves the retention/stabilization of the skin of the scrotum and penis.
The inner face has, on the side of the upper surface portion, one at least among: an axial extension offset axially towards the front relative to the rest of the inner face; and two projections spaced at least 5 mm apart and facing each other, on either side of a virtual median plane of the device, the two projections projecting radially inward.

According to a specific feature, locally the two projections form a same device's extra thickness, where a maximum thickness of the device, at least on the upper side of the device, is obtained.

According to a specific feature, the protrusions have a hollow and/or are a suction-cup shape. At least for a part of the protrusions, they may be wider than high, while also typically having a height of around 1 mm (each protrusion being significant, fully visible individually to the naked eye).

More generally such protrusions, sufficiently marked, form large interstitial spaces compliant with perspiration.

In an optional embodiment, the ring body has a notch for passage of the scrotal skin over the front edge side.

According to a specific feature, the median plane separating the device in two halves is a plane of symmetry, preferably a vertical plane of symmetry cutting/passing through the upper surface portion and the inner grip surface.

Optionally, the two projections form compression points, by serving to stabilize and prevent sliding of the testicles (sliding which could lead to their descent in the scrotum). They improve maintaining of the testicles in supra-scrotal position by the action of the ring forming body.

The ring, by firmly enclosing the penis and scrotum, exerts an upward counterforce serving to keep the testicles and the intended position. Likewise as it relates to the part of the scrotum which consequently cannot be inserted into the ring (part of the perineal zone), it is observed that it is tensed and held empty by the counter pressure exerted by the ring holding the penis and the scrotum.

The option with the two projections (forming restraining/compression points) advantageously limits the risk that the testicles could just the same migrate towards the perineal zone (poorly placed ring, sports practice, etc.), driving a slight sliding of the ring on the penis and the scrotum and therefore releasing the exerted counter pressure. Indeed, the projections contribute to barring the route to the testicles by exerting a pressure on the paths for descent around the ring towards the perineal zone.

Optionally, the material constituting the device is impermeable, preferably hydrophobic. This hydrophobic property typically serves to define a contact angle with liquid water (water in droplet/drop form) over 100°. This angle, for example, is of order 110-111° for a silicone type material.

According to a feature, the flexible material body forming the ring is solid, being free of internal cavities between the inner face and the outer face.

As a nonlimiting example, the average thickness of the material in the body forming the ring may be included between 1.5 and 7 mm, preferably between 2 and 5 mm. Thus, the external bulk of the device around the shaft is minimized.

The traction strength of the material may be at least 5 MPa. Further, a use of the device in order to easily place, hold and provide the effectiveness of the contraceptive device by displacement of the testicles is possible.

More specifically, the device is used in order to provide male contraception by encircling a shaft and the scrotal skin of the empty scrotum, in particular during a daily time greater than or equal to 14 or 15 hours, such that the protrusions of the inner face are in contact with the scrotal skin and exert a pressure upward or against gravity, which keeps the testicles in a proximal position near the inguinal canal (meaning in supra-scrotal position, near the root of the penis).

The raising effect or antigravity effect of the device, operating as an exoprosthesis, prevents the testicles from moving away from the warm zones of the body (which induces a heating of these testicles as compared with a lower position).

According to a specific feature, interstitial zones extend between the protrusions (which may greatly ease sweating/perspiration), such that the device is particularly suited for prolonged wearing, preferably for a time over 12 hours.

Putting it on is simple: it can be done seated, standing, lying on the back, lying on the side. The testicles position keeping is even described as agreeable and pleasant by many users.

An option for putting the device on may consist, for the user, of lying on the back and sliding the device around the shaft, along a direction equal or similar to the direction of gravity, and then raising the scrotal skin (empty scrotum) into the inner volume formed by the annular body of the device and/or in a slit of the retaining means. Whether in standing position or seated position, the device holds and supports from below the scrotal skin inserted in the inner volume or in the slit, respectively, because of the surface portion for the scrotal contact (surface portion which is part of the inner grip surface or which delimits the slit).

A local excess thickness of the device in this zone may make the effect of supporting the empty scrotum easier.

Typically, the effectiveness of the contraceptive effect is verified by routine, for example every 3 months, sperm analysis, called spermograms. Before wearing the ring, it may be timely to do an analysis for verifying the fertility of the person.

It is expected that the ring will be worn for around 15 hours per day without interruption. This length is indicative, knowing that an exceptional day with a lower wearing time is not problematic.

The inner diameter of the device may be selected from several sizes, for example three sizes, or at least five or six sizes. The usual categories can be found: “S” for small diameter that does not exceed 30 or 33 mm, “M” for medium diameter (of order 35 or 40 mm) and “L” for a large diameter of order 43 or 45 mm. An “XL” category for a very large size may for example correspond to a diameter of about 50 mm. An “XXL” category may also be provided, for example with an inner diameter of 52 or 53 mm. Analogously, an “XS” category for a very small size may for example correspond to a diameter of about 25 or 26 mm. An “XXS” category may also be provided, for example with an inner diameter of about 23 mm. In any case, the inner diameter will typically be included between 20 and 55 mm, preferably between 23 and 52 mm.

By choosing the most appropriate diameter, wearing this device is easily tolerated. The male contraceptive device may be compact and provided with tightening or compression points, in order to be compatible with a large number of activities, including the most physical or which demand the most unusual movements. As for the position lying on the stomach, which can be less agreeable, it remains unusual and is not taken for long while the user is awake.

A process of manufacturing the male contraceptive device according to the invention is also proposed by using a mold, wherein the process comprises the steps consisting essentially of:
providing a crosslinked/thermoset polymer, preferably silicon-based;
hot molding the thermoset polymer in ring shape in an annular cavity of the mold, while forming protrusions on the inner face by molding in corresponding hollow imprints of the mold located to face an outer peripheral part of the mold; and
cooling said material in order to allow cooling thereof and obtaining a solid and flexible state of the device.

The thermoset polymer may be a highly stretchable type, for example stretchable over 400 or 500% before rupture, which easily allows getting a very oval shaped stretched shape of the device. Typically, the stretching may be chosen in the range from 600 to 1000% (DIN 53 504 S2 standard). This greatly limits the risk of constriction when periodically wearing the device, for example about 15 hours per day.

The device may optionally be obtained by injection molding of a ready to use mixture (of components A and B). As is well known, these components are directly added to the molding machine by injection from the initial containers by means of a metering and mixing unit. The mixture, the compound of the two components in a ratio 1:1, is injected into the heated mold. As an example, the heating power corresponds to mold temperatures of 170 to 230° C.; the silicone rubber crosslinked by addition vulcanizes in a few seconds, without dissociation products.

The high setting speed and the easy unmolding allows an entirely automated production of a large number of items in short cycle times.

As an example, a material may be used resulting from a mixture of two elastomer or silicone components (optionally each having a similar viscosity, for example of around 70 in the meaning of the DIN 53018 standard, at 20° C.). The vulcanized/crosslinked mixture may have a density of 1.07 g/cm³ and a hardness of around 8 (Shore A hardness).

The device may be designed based on Silopren LSR 2010 TP 3740, the material thus being a standard silicone rubber obtained by bi-component injection molding.

More generally, the material may be silicone-based and have a traction strength included between 1.9 and 5 N/mm² (according to the DIN 53 504 S2 measurement standard).

Optionally, the tearing strength may be included between 3 and 13 N/mm, preferably between 4 and 9 N/mm.

### BRIEF DESCRIPTION OF THE DRAWINGS

Other features and advantages of the invention will become apparent during the following description of several of the embodiments thereof, given as nonlimiting examples, with reference to the attached drawings, in which:

FIG. 1 is a perspective view of a male contraceptive device in accordance to a first embodiment of the invention;

FIG. 2 is a side view of a male contraceptive device in accordance to a second embodiment of the invention;

FIG. 3 is a perspective view of the device from FIG. 2;

FIG. 4 is a bottom perspective view of a device according to a third embodiment similar to the one from FIG. 3;

FIG. 5 is a front view of a male contraceptive device whose front and rear surfaces are identical, according to a fourth embodiment;

FIG. 6A is a view of half of a male contraceptive device in an option with the lower part inclined from front to back, which serves to form a larger passage section from the side of the rear edge compared to the passage section defined by the front edge;

FIG. 6B is a view of half of a device similar to the one from FIG. 2 serving to show the sections in the annular body of the device;

FIG. 7 is a side view showing a male contraceptive device which is distinguished in particular from the second embodiment by a notch on the side of the front surface;

FIG. 8A shows a male contraceptive device conforming to another embodiment of the invention, with a slit separating two subelements of the lower part of the device;

FIG. 8B is a view, along a vertical median section, of half of the device from FIG. 8A;

FIG. 9A is a perspective view of a male contraceptive device conforming to another embodiment of the invention, with a space separating two layers of the lower part of the device;

FIG. 9B is a view, along a horizontal median section, of an upper part of the device from FIG. 9A;

FIG. 10 is a vertical section view showing a detail of the device from FIGS. 9B and 9B.

In the figures, same references designate identical or similar items.

### DETAILED DESCRIPTION OF EMBODIMENTS

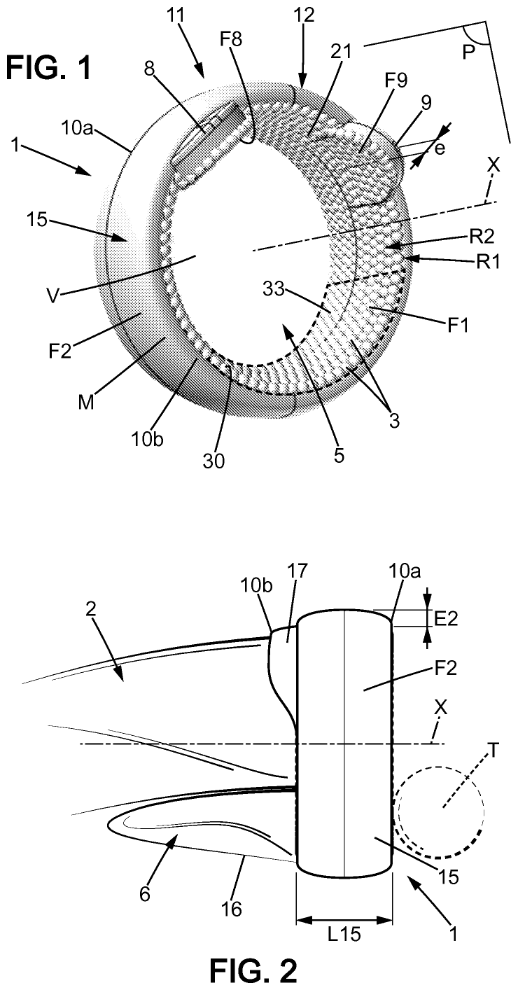

Referring to FIGS. 1, 2 and 3, the male contraceptive device 1 has a generally annular shape with which to encircle the shaft 2 of the wearer. The inner face F1 of the device 1 has irregularities resulting from protrusions 3. An inner grip surface 30, typically continuous, may be made up with such protrusions 3. In this nonlimiting example, the device 1 extends continuously around a virtual longitudinal axis X. Such an axis X may optionally belong to a median plane of symmetry P which separates the device 1 into two halves 11, 12.

The outer face F2 of the device 1 may extend between two opposite axial ends. The outer face F2 is for example smooth and forms continuously rounded circumference. Here, the two axial ends may be defined by two respective annular edges 10a, 10b. The rear edge 10a is designed and positioned to come stop against the side of the root of the shaft 2 in the worn state of the device 1 with the shaft 2 encircled.

As shown in FIG. 2, the male contraceptive device 1 serves to encircle the shaft 2 and the empty scrotum 6 of the wearing individual of childbearing age. A rear annular edge 10a, which may extend transversely to the longitudinal axis X (perpendicular placement in the case of the FIG. 2) of the device 1, comes to stop at the side of the root of the shaft. The inner face F1, which delimits an inner volume V of the device 1 has protrusions 3 uniformly distributed or closely spaced at least in the area of the inner adhering/grip surface 30.

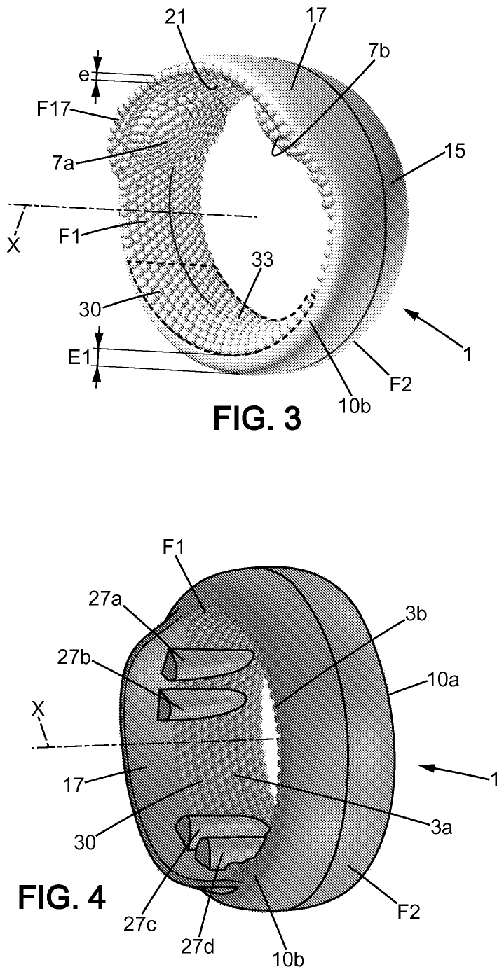

The top of the shaft 2 may engage against a concave upper surface portion 21 of the device 1, as can be seen in FIGS. 1 and 3. Opposite this upper surface portion 21, the inner grip surface 30 has an anti-slip surface portion 33.

In the example from FIGS. 1 to 7, this anti-slip surface portion 33 serves for the scrotal contact, and is in practice separated from the shaft 2 in order to allow holding of the scrotal skin portion in a lower zone of inner volume V with direct contact against the protrusions 3. Since the spacing between the shaft 2 and the facing protrusions 3 is sufficiently small, the empty scrotum 6 is held tight in the inner volume V without it being possible for the testicles T to enter into this volume V. In fact, the rear annular edge 10a pushes the testicles T backward from the inner volume V.

The surface portion 33 for the scrotal contact, defined straight below the upper surface portion 21 in order to provide pressure on the empty scrotum 6, corresponds to a surface area of at least 2 or 3 cm².

In the embodiments shown, the inner adhering surface 30 extends over the majority or the entirety of the inner face F1 such that the protrusions 3 are present in zones opposite each other.

Here, the upper surface portion 21, concave, of the inner face F1 allows contact with the shaft 2 from the side of the superficial dorsal vein thereof while providing a guiding and stabilizing effect. Protrusions 3, here in the form of nubs, may be provided in this upper surface portion 21. The inner face F1 may be locally extended (preferably forward) and/or provided with support or compression points, which serve to increase the contact zone between the shaft 2 and this upper surface portion 21.

An anti-slip effect may be obtained from the formation of protrusions 3, typically nubs laid out in parallel rows R1, R2, in combination with a relatively low hardness of the material M forming the device 1 (for example a hardness less than or equal to 30 on the Shore A scale). This anti-slip effect may be provided at least in the upper surface portion 21 in order to limit the risk of slipping of the device 1 during phases of movement of the wearer (e.g. walking, running, jumping, etc.).

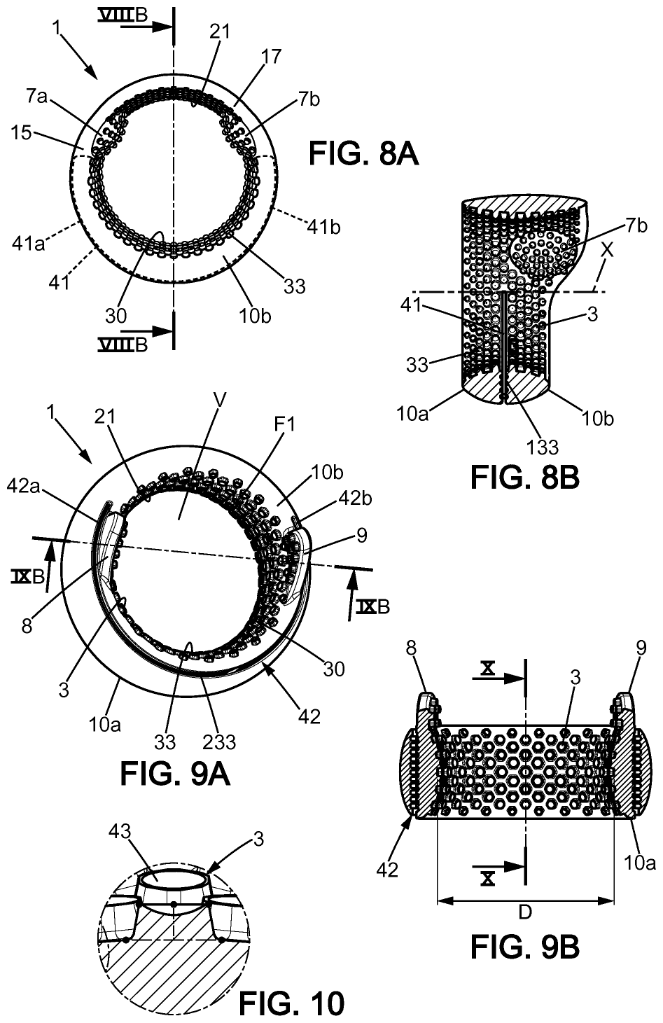

In the options shown in FIGS. 1 to 7, the device 1 may also comprise the same type of protrusions 3 in order to form the inner grip surface 30 and the upper surface portion 21. Alternatively, at least two groups of structurally different protrusion may be provided. As a nonlimiting example, a roughness resulting from submillimeter unevenness can be provided in one zone, while the other zone has nubs having an extension of at least 1 mm. In other variants, like those shown in FIGS. 8A to 9B, a group of protrusions is formed inside in a slit 41 or 42 in order to form an adhering zone which extends parallel to the inner grip surface 30.

Preferably, several tens of nubs, each visible to the naked eye, are formed in order to form the grip surface 30. In the examples from the Figures, the protrusions 3 are typically nubs laid out in parallel rows which each completely circle the longitudinal axis X. As can be clearly seen in FIGS. 1 and 3, these protrusions 3 are here distributed from one edge to the other 10a, 10b at least in the lower part of the device intended for contact with the empty scrotum 6.

As shown in FIG. 1, the inner volume V which is delimited by the inner face F1 may correspond to a volume with substantially cylindrical or continuously rounded contour, in an undeformed state of the device 1. The longitudinal extension of this inner volume V is limited and typically less than the largest inner width or diameter D defined by the through passage 5 (central passage surrounded by the inner face F1). This inner volume V has a width typically less than or equal to 50 or 53 mm but greater than 27 mm such that it may be filled by a section of the shaft 2 and by the empty scrotum 6.

To make this insertion of the shaft 2 through the central through passage 5 easier, the inner face F1 may have a generally circular circumference with possible exceptions, in particular when one or more projections 7a, 7b are provided as in the case shown in FIG. 3.

The projections 7a, 7b protrude for example inward (relative to the rest of the inner face F1) with a height of at least 1 or 2 mm and/or, typically, may have an extension dimension towards the longitudinal axis X which is greater than the height of the protrusions 3. These projections 7a, 7b may each optionally include other protrusions 3 in the form of nubs, as in the remainder of the inner face F1.

The projections 7a, 7b limit one of the types of descent of the testicles T. The path for possible bypass of the ring is in fact blocked by the projections 7a, 7b preventing the testicles T from migrating from the perineal zone. The projections 7a, 7b are flexible, typically with a large capacity for deformation in order to adapt to the morphology of each user and to different movements and postures.

In the nonlimiting example from FIG. 3, the spacing between the two projections 7a, 7b, for example included between 14 and 40 mm, is such that a throat zone is formed in an upper portion 21 of the inner grip surface 30. Optionally, these two projections 7a, 7b may form compression points which are adjacent to the rear edge 10 (of the side of the root of the shaft) and are located:
on either side of the median plane P, which could be a plane of symmetry;
one in an angular section which may be included between 22 and 67° and the other in another angular section which may be included between 292 and 337° (knowing that the 0/360° reference corresponds to the upper point of the inner face F1 of the device 1, meaning the center of the upper surface portion 21 facing the superficial dorsal vein of the penis.

Now referring to FIG. 4, more than two upper projections can be seen. Here four projecting members 27a, 27b, 27c, 27d are formed on the inner face F1, each with a narrowed and/or tapered zone in order to define a leakage edge oriented towards the rear and possibly placed away from the rear annular edge. In order to get a stabilizing effect, the surface of these projecting members 27a, 27b, 27c, 27d may be uneven. These projecting members may be gathered and brought together in pairs in order to be located, in the inner face F1, similarly to the aforementioned location in the case of the two projections 7a, 7b.

Whether it is with the device from FIG. 3 or the one from FIG. 4, the throat shaped spacing (between the projections/projecting members) in the portion of the upper surface 21 may help the device to be deformed by making it slightly less curved from the side of the upper surface portion 21. This deformation may serve to better distribute the retaining force from the upper side, by also holding the scrotal skin 16 laterally (which results from a slight inverted trapezoidal deformation effect). In other words, the separation of the compression/restraining points may correspond to an optimal position for support of the testicles without compressing the penis.

The material constituting the device 1 is weakly compressible or incompressible and has a density at least equal to 1, for example at least equal to 1.04. This is a flexible material M which has a significant extensibility. When the device 1 is made of a single material part, this material M may have anti-sliding properties. It typically involves a silicone.

Of course, the flexible material M of the device 1 may be stretched without risk of tearing, in particular in radial directions, knowing that the lengthening thereof before rupture is at least 400%, for example, preferably from 650 to 1000%.

FIG. 1 shows an option in which the device 1 comprises a pair of projecting elements 8, 9 projecting towards the front side relative to the body 15 in form of regular ring/constant axial extension. These projecting elements 8, 9 extend past for example the maximum of 5 mm forward relative to the remainder of the front edge 10b. Here each of the projecting elements 8, 9 has a frontal portion for which the profile is “C”-like by thus forming a continuously convex front surface from one of the junctions with the circular part of the front edge 10b to the other. Other profiles, for example forming waves, may also be used. The projecting elements 8, 9 define axial extensions F8, F9 from the inner face F1 serving to further stabilize the device 1.

The protrusions 3 may be present on the inner side of these projecting elements 8, 9 but not on the front surface with the possible exception of a row or edge of protrusions 3b projecting forward from the front surface defined in the corresponding element 8 or 9.

The thickness e of the projecting elements 8, 9 or of any other analogous extension 17 may be less than the thickness E2 of the body 15 in the upper part, for example by being two times smaller than the thickness E2. Referring to FIG. 3, the axial extension F17 which extends forward from the inner face F1, from the upper side, is concave by following the general curvature of the front edge 10b. The radius of curvature of the projecting element 17 may thus be the same as in the main body 15 of the device 1.

It is understood that the projecting elements 8, 9 and the protrusions 3, here distributed in several facing zones on the inner face F1, improve the general hold of the device 1 to the root of the shaft 2. The protrusions 3 may be directly integrated with the flexible material M constituting the device. In variants, the flexible material M is not the sole component of the device 1 and may only form an added/overmolded coating on another layer of flexible and stretchable material.

Further, as can be seen in FIG. 9A, using a slit 42, a radial expansion space may be formed, which extends between two subelements each forming a respective layer of the ring. The layer proximal to the longitudinal axis X may thus have an increased elastic deformation, which provides the user extra comfort. The slit 42 for defining such a space may be obtained during molding of the silicone body. Such a slit 42 may be provided in a wide range of devices conforming to the invention. Alternatively, a slit 41 may be provided which extends through the body of device 1 for the most part in a plane perpendicular to the longitudinal axis A as in the case shown in FIGS. 8A and 8B. This design may also allow an expansion with effect of separation between the edges 10a and 10b.

The FIGS. 2 and 3 show an option with a single projecting element 17 larger than the elements 8, 9 and which is distributed in the two halves 11 and 12. This type of projecting elements 8, 9, 17 may be formed from the upper side of the device 1, in order to engage with the upper side of the shaft 2 on both sides of the superficial dorsal vein. When such projecting elements 8, 9, 17 are present, the protrusions 3 of the inner face F1 may also be provided on the inner side of these projecting elements 8, 9, 17.

Having such projecting elements 8, 9, 17 allow the inner face in contact with the shaft 2 to be locally extended. More generally, it is understood that the inner face F1 may have, preferably on the side of the upper surface portion 21, at least one among:
an axial extension (resulting from a projecting element 8, 9 or 17) axially offset forward relative to the remainder of the inner face F1; and
two projections 7a, 7b separated from each other and facing each other from both sides of the virtual median plane P of the device 1, where the two projections 7a, 7b are radially projecting inward.

In a part of the device 1 complementary to that which comprises the extension 17 or the elements 8, 9, a space or a slit 41, 42 which extends transversely over a portion of the periphery of the ring, as shown for example in FIGS. 8A and 9A, may be provided.

In a variant, the device 1 may consist of a ring having a symmetry of revolution around the longitudinal axis X. In this case, the device 1 may be formed uniquely by the body 15 shown in FIG. 1 without the projecting elements 8, 9.

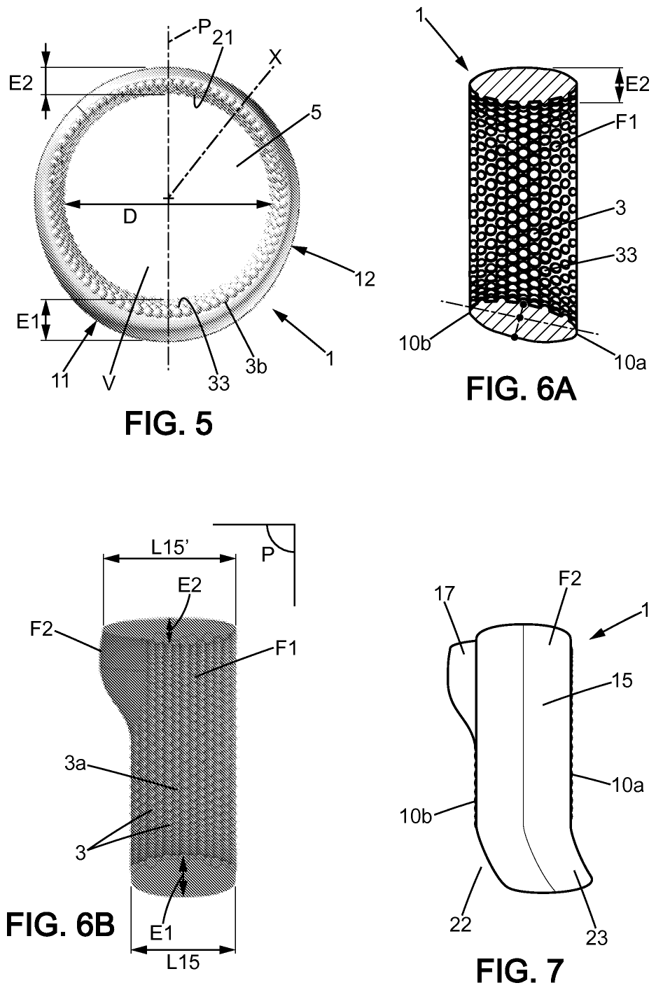

Further, as shown in FIGS. 5 and 6B, the outer face F2 and the inner face F1 may have a variable thickness between them. The thickness E1 measured in the lower part of the device 1, here in median plane P, may be greater, for example greater with at least 1 mm thickness excess, than the thickness E2 measured in the upper part of the device 1, here in the median plane P.

Optionally, the device 1 breaks down into two identical halves 11, 12 separated from each other by the median plane P, and may further not have any portions projecting forward or rearward. The device 1 could have a front and a rear surface which are identical. This makes it possible to place the device 1 on the shaft 2 without risk of mistaking the insertion direction.

In these embodiments, the protrusions 3 of the inner face F1 may have the shape of nubs, for example the shape of half-beads entirely formed with the layer forming the inner face F1. Although the figures show an inner face F1 provided continuously with protrusions 3, areas without protrusion may also be provided.

At least two series of protrusion may be provided, structurally different or not. In the example from FIG. 1, the edge protrusions 3b are distinguished from the other protrusions 3a in that they also project forward or rearward relative to the outer face F2, respectively depending on whether they are located along the rear edge 10a or along the front edge 10b.

The semi-beads or similar nubs covering the inside of the ring-shaped body 15 improve the retaining/stabilization of the skin of the scrotum 16 and skin of the penis. They serve to improve keeping the testicles T in supra-scrotal position at the root of the shaft near the outer orifice of the inguinal canal, in order to induce an increase in temperature of the testicles with the human body as the only heat source.

On FIG. 1, protrusions 3 are shown arranged in respective rows R1, R2 and they are optionally staggered. With this type of structure for forming the inner face F1, it is of interest to define at least three or four parallel rows of protrusions. In this nonlimiting example, each row R1, R2 is arranged to be generally parallel to the edges 10a and 10b.

The axial extension L15 of the body 15 is between 15 and 24 mm and may be constant (see distance measured longitudinally between the two edges 10a, 10b) except in the upper part where optional projecting elements 8, 9, 17 are provided (with a locally larger axial extension L15′). When the axial extension L15 is over 15 mm, at least seven or eight different rows, or at least seven or eight protrusions or more may be provided placed on a longitudinal segment going from the rear edge 10a to the front edge 10b.

Referring to FIGS. 5 and 6A-6B, it can be seen that the inner face F1 of the body 15 may have a constant width (corresponding to the axial/longitudinal extension L15 and typically chosen between 15 and 24 mm) but less thickness on the upper side of the device 1. The reduced thickness E2 serves to contribute to the deformation and the comfort of wearing. The Shore A hardness of the material making up the inner face is included between 8 and 30, preferably between 10 and 20.

This type of structure and the soft contact makes the device 1 particularly well suited for playing regular or intensive sports. The additional presence of projections 7a, 7b like those that can be seen in FIG. 3 is also compatible with playing sports.

In the options with a symmetry relative to the plane P, the virtual longitudinal axis X constitutes a central axis equidistant between the upper surface portion 21 and the surface portion 33. Optionally, as can be seen in FIG. 6A, a widening of the axis opening from the side of the rear edge 10a may be provided. Here, the rear edge 10a goes down lower than the front edge 10b because of the inclination of the lower part of the body forming the device 1.

A thickness of the penis-scrotum ring, which increases progressively away from each of the annular edges 10a, 10b (thickness gradient in the ring), can also be provided, for example with the maximum thickness substantially at the midpoint between the two axial annular ends. The thickness E1 may be the same, with a single profile, which is of interest for forming a ring which is worn in both directions, with interchangeable edges 10a, 10b in order to form the front and rear.

Further, the protrusions 3 of the device 1 may have a larger section away from the two edges 10a, 10b. This increase of section makes the device 1 comfortable even with a large height of the protrusions 3, for example greater than or equal to 0.9 or 1 mm. The interstitial zones between the nubs or protrusions 3 can make perspiration easier, which makes the device 1 particularly suited to extended wearing, typically for a time of over 12 hours.

Compared to the device from FIG. 5, the devices from FIGS. 1, 2, 3, 4 and 7 have the advantage of better distributing the pressure on the top of the shaft 2, because of the elements 8, 9, 17 projecting in the forward direction. The fact of extending the inner face F1 with axial extensions supplements the deformable nature and provides a major advantage in comfort for people spending long times seated while wearing the device 1.

The characteristic dimension of the through passage 5, here an inner diameter D, may be of about 40 mm. For comfortably wearing the device 1, it may be attractive for the user to select their size, for example among diameters D of 30, 40 or 50 mm.

Now referring to FIGS. 8A and 8B, it can be seen the upper part of the device 1, thicker, may optionally have a slit 41 passing therethrough.

Independently of the way of implementing the protrusions 3 and forming the upper part, the slit 41 may be of interest in that it forms a passage allowing insertion of an end of the empty scrotal pocket (part of the scrotal skin) with a retaining effect fairly comparable to the retaining of a strap, for example. The elasticity of the material of the device 1 serves to separate this slit 41 at the time of insertion. If this end is slid through the slit 41 in order to come out below the side of the end surface F2, the separated slit 41 is then re-closed and risk of the scrotal skin portion 16 coming back out by the rear of the inner volume V is minimized.

Of course, with this device 1 provided with a slit 41, the user may just as well choose to place the scrotal skin portion 16 in the slit 41 or in a lower part of the inner volume V as in the case shown in FIG. 2. The ends 41a, 41b of the slit 41 are separated from each other, hereby being diametrically opposite which allows adaptation to a wide variety of scrotal skin 6.

As shown in FIG. 8B, the inner faces delimiting the slit 41 may be ribbed, grooved or textured to improve the adhesion against the scrotal skin portion 16 for better retention. More generally, any retaining means may be provided in order to form the slit 41 and define one or more surface portions 133 against which the scrotal skin 6 is engaged with an adhesion force.

Referring to FIG. 9A, it can be seen the projecting elements 8, 9 may be placed at mid-height on the device 1, where these projecting elements are optional and interchangeable with the extension 17 or similar extension as shown in FIG. 8B. Further, a slit 42 can be provided defining a radial expansion space in the lower part of device 1, shown here in the form of a slot going from the front edge 10b to the rear edge 10a. This type of slot allows a mobility of the lower part of the device 1 with an expansion effect in a radial direction.

This type of radial displacement may accompany a natural expansion of the scrotum.

The scrotal skin 6 may selectively be slid through this slit 42 from the rear edge 10a to come out from the side of the front edge 10b. The slit 42 which is separated during this insertion of the scrotal skin 6 may then close up and the risk of the scrotal skin portion 16 coming back out by the rear of the inner volume V is minimized.

More generally, any retaining means may be provided to form the slit 42 and define one or more surface portions 233 against which the scrotal skin 6 is engaged with an adhesion/grip force.

Of course, with this device 1 provided with a slit 42, the user may just as well choose to place the scrotal skin portion 16 in the slit 42 or in a lower part of the inner volume V as in the case shown in FIG. 2.

As illustrated in FIG. 9B, the inner faces delimiting the space 42 may be ribbed, grooved or textured in order to improve the adhesion against the scrotal skin 6 for better retaining. In the example shown, the slot forming the space 42 has two opposite ends 42a, 42b, which extends beyond the horizontal median plane (and above the projecting elements 8, 9). The significant gap between the ends 42a, 42b of the slit 42, larger than the inner diameter D makes insertion of the scrotal skin 6 easier for a wide range of sizes/geometries of this scrotal skin.

In the exemplary embodiment of FIGS. 9A and 10, it is provided an option, applicable to any device 1 in accordance to the invention, according to which the protrusions 3 have a hollow 43 and/or a suction-cup shape. A very good adhesion is obtained that way, with protrusions 3 which is similar to the suction cups present on the arms of an octopus.

At least for some protrusions 3, the lateral surface delimiting the hollow 43 may be non-cylindrical, a conic section which flares towards the contact surface of the protrusions 3.

An advantage of the device 1 is the compactness thereof and the comfort of using it. It is well suited to providing male contraception once it is worn around 14 or 15 hours per day, where the TMC contraceptive effect (thermal male contraception) can be observed after 2 months of regular wear, analogously to what was verified by the work of Prof. Mieusset in the context of wearing a brief raising the testicles (Roger Mieusset and Louis Bujan: The potential of mild testicular heating as a safe, effective and reversible contraceptive method for men, International journal of andrology, 17:186-191, 1994).

The device 1 alone and without medical or surgical intervention constitutes an effective tool for contraception. The reversibility of the method and the absence of side effects are further significant advantages for a low-cost device 1.

The annular male contraceptive device 1 may be produced industrially by hot molding, in a mold which may typically be annular. The device 1 may be free of latex and be hypoallergenic. Preferably, it contains neither colorants nor bisphenol A, nor phthalate compounds, nor bleaching agents, nor toxins.

The production process for the device 1 may use a polymer, thermoset polymer for instance, in order to get a shape memory effect, such flexible material M being preferably silicone-based. The thermoset polymer is hot mold in ring shape in an annular cavity of the mold, while forming protrusions 3 on the inner face F1 by molding in corresponding hollow imprints of the mold. Such imprints are located facing an external peripheral part of the mold. The external surface could be formed smooth, without need for special imprints on the outer peripheral part of the mold.

The molding is typically injection molding. After cooling of the molded material, the device 1 results which is solid and has a relatively low hardness, where the material M is flexible and stretchable.

One or more surface treatment steps may be done, for example after solidification. The inner face F1 and the outer face F2 may in that way be made softer to the touch.

In an implementation variant, the device 1 may be obtained by an additive production technique. 3D printers from Wacker are suitable, for example, for using silicone as 3D printing material.

In this case, printing instructions are provided from a file representative of spatial coordinates of the device 1 (coordinates in a typically Cartesian reference frame). The silicone injector is next moved along a routine for reconstituting the successive layers with which to form the device 1. In the case of a flexible material M of silicone type, it may be deposited drop by drop with a high viscosity. The printing support or bed may be a glass support. At the end of depositing each layer, the droplets are fused by vulcanization with a UV source. This production mode may be well suited when there is a need to implement small production runs or prototypes.

As for the precise shape, the diameter D or analogous dimension, position of compression points, values or information as above provided have been specified solely for a non-limiting way of making the device 1.

In a variant implementation, apparent from FIG. 7, the body 15 of the ring has a notch 22 for the passage of the scrotal skin 16 from the side of the front edge 10b. The notch 22 extends for example, from front to back, over a distance less than or equal to 10 mm, preferably less than or equal to 5 or 6 mm. A projection 23 may be formed on the rear edge 10a of the lower side, in order to avoid too much reduction of the surface portion 33 for the scrotal contact.

Further, accessorizing the device 1 with an appendage may be planned, for example an appendage, vibrating or not, for the clitoris. The appendage is either integrally formed with the body 15 by being joined to the outer face F2, or removably attached on an appropriate attachment member formed on the outer face (of the lower side of the device).

In some options, the device 1 may form a connected object or have a timer with which to determine the wearing time. With an electronic communication accessory, the user could connect/pair the device 1 with an application, typically an application downloadable over the Internet, in order to manage their contraception (time, effectiveness, break time, discussion forum for practices, etc.).

The contraception device 1 (control of the fertility) advantageously eliminate active heating zone: the supra-scrotal position of the testicles T, in contact with the body heat, is sufficient for getting the desired effect. It is therefore a self-heating constant temperature device 1 for personal use.

Thus, even though the examples shown illustrate a device 1 in the form of a ring made of one part or constantly closed, the device 1 may as needed form a loop that can be opened. In an option, the closed loop configuration is allowed with an adjustment of the length of the inner perimeter of the annular device.

The device 1 may optionally have the shape of an adjustable belt. Alternatively, an elastic hinge and closure means (including mechanical elements, for latching, self-adhering members, magnetic elements, other similar elements or combinations of these means) may be provided, with typically a lock for the loop configuration of the device 1.

A plastic part for which the hinge has a bistable nature, which may allow changing closure means or limiting the closure contact to a superposition between the ends of the device, could be used.

1. A male contraceptive device adapted to encircle a shaft, the device having an annular shape around a virtual longitudinal axis and comprising:
two annular edges forming two opposite axial ends of the device, a first edge of the two edges being a rear abutment edge on the side of the shaft root in mounted state of the device with the shaft encircled;
an outer face;
an inner face delimiting an inner volume and having protrusions to form an inner grip surface of the device, the inner face comprising an upper surface portion, concave, intended to be in contact with the shaft on the side of superficial dorsal vein thereof;
wherein interstitial zones are distributed on the inner face, between the protrusions, to facilitate perspiration;
wherein the device further comprises a surface portion for scrotal contact, adapted to be spaced from the shaft and located opposite the upper surface portion, such that it enables to keep a scrotal skin portion between the shaft placed in the inner volume and the surface portion for scrotal contact, whereas said rear abutment edge pushes back the testicles behind the inner volume,
and wherein the surface portion for scrotal contact is formed at least in part by said inner grip surface and/or by a lower retaining portion forming a slit which extends under the inner grip surface.

2. The device according to claim 1, wherein the inner face has an annular configuration enabling to define a through passage whose radial extension measured perpendicularly to the longitudinal axis, is at least 27 mm and at most 53 mm in an undeformed state, the through passage delimited by the inner face is deformable by elastic deformation of a flexible material forming the device, the distance between the two annular edges is comprised between 15 and 24 mm at least on the side of said surface portion for scrotal contact.

3. The device according to claim 1, wherein the protrusions are in the form of nubs, the device comprising a flexible polymer material to define the nubs.

4. The device according to claim 1, wherein the surface portion for scrotal contact corresponds to a surface area of at least 2 cm² and has first protrusions which project radially inward.

5. The device according to claim 1, wherein the device is in the form of a ring made as a single-piece, in an elastomer.

6. The device according to claim 1, comprising a structure with at least two layers in a lower part of the device, the protrusions of the inner face being at least in part formed by a layer referred as inner layer defining a coating, said slit being formed between two layers extending under said inner layer.

7. The device according to claim 1, wherein the inner face has, on the side of the upper surface portion, at least one among:
an axial extension axially offset forward relative to the remainder of the inner; and
two projections spaced at least 5 mm apart and facing each other, on either side of a virtual median plane of the device, where the two projections project radially inward.

8. The device according to claim 1, wherein the retaining lower portion forming a slit is part of an annular piece constituting the device.

9. The device according to claim 8, wherein the retaining lower portion delimits a housing, distinct from the inner volume, which is formed internally in a lower part of the device to receive the scrotal skin portion, the housing being delimited by two facing surfaces which present retaining reliefs.

10. The device according to claim 8, wherein the slit is formed transversely to the longitudinal axis by having a first opening in the inner face and a second opening, opposite the first opening, in the outer face.

11. The device according to claim 1, wherein the inner face defines an inner diameter measured perpendicularly to the longitudinal axis, where the slit extends between two ends separated from each other by a distance greater than the inner diameter.

12. The device according to claim 1, wherein the device serves to encircle a shaft and the scrotal skin of the empty scrotum to provide a male contraception, during a daily time greater than or equal to 14 hours, whereas the protrusions of the inner face are in contact with the scrotal skin so as to keep the testicles in a proximal position near the inguinal canal.

13. A process for manufacturing the male contraceptive device according to claim 1 by using a mold, the process comprising:
providing a thermoset polymer;
hot molding the thermoset polymer in ring shape in an annular cavity of the mold, while forming protrusions on said inner face by molding in corresponding hollow imprints of the mold located to face an outer peripheral part of the mold; and
cooling said material in order to allow hardening thereof and obtaining a solid and flexible state of the device.

14. The device according to claim 1, wherein at least eight of the protrusions are distributed along a single circular trajectory in a transverse section of the device, over a half for contact with the scrotal skin.

15. The device according to claim 14, wherein the inner face is provided with at least eight nubs per square centimeter.

16. The device according to claim 14, wherein on the surface portion of the inner face for the scrotal contact, maximum inter-protrusion distance is less than 7 mm.

17. The device according to claim 14, wherein the protrusions have a height, measured perpendicularly to the longitudinal axis, of at least 0.4 mm,
and wherein the protrusions are distributed in parallel transverse rows with each transverse row extending over more than half of inner circumference of the device, a network of protrusions being formed with longitudinal rows of at least three protrusions along the longitudinal direction.

18. The device according to claim 17, wherein the protrusions are forming the grip surface 30, the protrusions comprising several tens of nubs.

19. The device according to claim 2, wherein the protrusions are in the form of nubs, the device comprising a flexible polymer material to define the nubs.

20. The device according to claim 2, wherein the surface portion for scrotal contact corresponds to a surface area of at least 2 cm² and has first protrusions which project radially inward.
